SEQUENCE LISTING (10 sequences)

SEQ ID NO: 1 - DNA, 29 bp, synthetic construct
  misc_feature 1..29: note = Synthetic Oligonucleotide
  source 1..29: mol_type = other DNA
  misc_difference 23: note = wherein n is a uracil
aatgatacggcgaccaccgaganctacac

SEQ ID NO: 2 - DNA, 24 bp, synthetic construct
  misc_feature 1..24: note = Synthetic Oligonucleotide
  source 1..24: mol_type = other DNA
caagcagaagacggcatacgagat

SEQ ID NO: 3 - DNA, 20 bp, synthetic construct
  misc_feature 1..20: note = Synthetic Oligonucleotide
  source 1..20: mol_type = other DNA
aatgatacggcgaccaccga

SEQ ID NO: 4 - DNA, 21 bp, synthetic construct
  misc_feature 1..21: note = Synthetic Oligonucleotide
  source 1..21: mol_type = other DNA
caagcagaagacggcatacga

SEQ ID NO: 5 - DNA, 29 bp, synthetic construct
  misc_feature 1..29: note = Synthetic Oligonucleotide
  source 1..29: mol_type = other DNA
aatgatacggcgaccaccgagatctacac

SEQ ID NO: 6 - DNA, 27 bp, synthetic construct
  misc_feature 1..3: note = diol linker subject to chemical cleavage
  misc_feature 1..27: note = Synthetic Oligonucleotide
  source 1..27: mol_type = other DNA
yyycaagcagaagacggcatacgagat

SEQ ID NO: 7 - DNA, 35 bp, synthetic construct
  misc_feature 1..35: note = Synthetic Oligonucleotide
  source 1..35: mol_type = other DNA
gctggcacgtccgaacgcttcgttaatccgttgag

SEQ ID NO: 8 - DNA, 35 bp, synthetic construct
  misc_feature 1..35: note = Synthetic Oligonucleotide
  source 1..35: mol_type = other DNA
cgtcgtctgccatggcgcttcggtggatatgaact

SEQ ID NO: 9 - DNA, 35 bp, synthetic construct
  misc_feature 1..35: note = Synthetic Oligonucleotide
  source 1..35: mol_type = other DNA
acggccgctaatatcaacgcgtcgaatccgcaact

SEQ ID NO: 10 - DNA, 34 bp, synthetic construct
  misc_feature 1..34: note = Synthetic Oligonucleotide
  source 1..34: mol_type = other DNA
gccgcgttacgttagccggactattcgatgcagc

(19) United States
(12) Patent Application Publication
(10) Pub. No.: US 2023/0116852 A1
(43) Pub. Date: Apr. 13, 2023

(54) METHODS FOR PREPARING SUBSTRATE SURFACE FOR DNA SEQUENCING

(71) Applicant: Illumina, Inc., San Diego, CA (US)

(72) Inventors: Hayden Black, San Diego, CA (US); Mathieu Lessard-Viger, San Diego, CA (US); James Tsay, San Diego, CA (US)

(21) Appl. No.: 17/813,852

(22) Filed: Jul. 20, 2022

Related U.S. Application Data
(60) Provisional application No. 63/225,081, filed on Jul. 23, 2021.

(51) Int. Cl. C12N 15/10; C12Q 1/6806

(52) U.S. Cl. CPC C12N 15/1068; C12Q 1/6806

(57) ABSTRACT

Embodiments of the present disclosure relate to method of preparing a substrate for sequencing by synthesis, including capturing library DNA to the surface using a low salt buffer solution prior to grafting primer oligonucleotides. Substrates prepared by the method described herein have increased monoclonality of clusters and sequencing by synthesis using the substrate prepared by the method are also described.

### FIELD

The present disclosure relates to methods of preparing substrate surface for sequencing applications, such as nucleic acid sequencing.

### REFERENCE TO SEQUENCE LISTING

The present application is being filed along with a Sequence Listing in electronic format. The Sequence Listing is provided as a file entitled Sequence_listing_ILLINC565A.xml created Jul. 14, 2022, which is 14.1 Kb in size. The information in the electronic format of the Sequence Listing is incorporated herein by reference in its entirety.

### BACKGROUND

Many current sequencing platforms use “sequencing by synthesis” (SBS) technology and fluorescence-based methods for detection. In some examples, numerous target polynucleotides isolated from a library to be sequences, or template polynucleotides, are attached to a surface of a substrate in a process known as seeding. Multiple copies of the template polynucleotides may then be synthesized in attachment to the surface in proximity to where a template polynucleotide of which it is a copy was seeded, in a process called clustering. Subsequently, nascent copies of the clustered polynucleotides are synthesized under conditions in which they emit a signal identifying each nucleotide as it is attached to the nascent strand. Clustering of a plurality of copies of the seeded template polynucleotide in proximity to where it was initially seeded results in amplification of signal generated during the visualizable polymerization, improving detection.

Seeding and clustering for SBS work well when as much of an available substrate surface as possible is seeded by template polynucleotides, which may maximize an amount of sequencing information obtainable during a sequencing run. In general, the less available surface area of a substrate used for seeding and clustering, the less efficient an SBS process may be, resulting in increased time, reactants, expense, and complicated data processing for obtaining a given amount of sequencing information of a given library.

A library of template polynucleotides may generally include a high number of template polynucleotide molecules whose nucleotide sequences differ from each other's. If two such template polynucleotides seed too closely together on a surface of a substrate (for example, an unpatterned surface), clustering may result in spatially comingled populations of copied polynucleotides, some of which having a sequence of one of the template polynucleotides that seeded nearby and others having a sequence of another template polynucleotide that also seeded nearby on the surface. Or, two clusters formed from two different template polynucleotides that seeded in too close proximity to each other may be too adjacent to each other or adjoin each other such that an imaging system used in an SBS process may be unable to distinguish them as separate clusters even though there may be no or minimal spatial comingling of substrate-attached sequences between the clusters. Such a disadvantageous condition may generally be referred to as polyclonality. For a patterned surface containing a plurality of confined compartments or location (such as a surface containing a plurality of nanowells separated by interstitial regions), polyclonality generally results from multiple seeding of different template polynucleotides in the same confined location and the subsequent amplification process produce more than comingled populations of copied of template polynucleotides in the same confined location. Seeding and clustering also work well when template polynucleotides from a library with different sequences seed on, or attach to, positions of the surface (e.g., an unpatterned surface) sufficiently distal from each other such that clustering results in spatially distinct clusters of copied polynucleotides each resulting from the seeding of a single template polynucleotide, a condition generally referred to as monoclonality. For a patterned surface, monoclonality refers to the condition when each compartment or confined area (e.g., nanowell) is seeded with a single template polynucleotide, or a single dominant template polynucleotide, such that clustering results in a single cluster of identical copies of the same template polynucleotide or a single dominant cluster in the same compartment or confined location. Polyclonality may result in lower library capture efficiency, higher noise to signal ratio during sequencing, and lower data quality. Therefore, it is desirable to perform SBS under conditions under which as much available surface area as possible of a substrate surface is used for seeding and clustering, while also promoting separation of seeded template polynucleotides so as to maximize monoclonality of clusters as possible and minimize polyclonal clusters. As such, there exist a need to develop new methods for improving monoclonality of the polynucleotides during the clustering process.

### SUMMARY

Disclosed herein are compositions and methods that may be used for improving monoclonal clustering in SBS.

Some aspect of the present disclosure relates to a method of preparing a substrate for sequencing, comprising:

contacting a first buffer solution comprising template polynucleotides with a surface of the substrate, wherein the surface of the substrate comprises a first plurality of bonding sites for capturing template polynucleotides and a second plurality of bonding sites for capturing primer oligonucleotides; and attaching the template polynucleotides to the surface of the substrate by forming covalent bonding or non-covalent bonding between the template polynucleotides and the first plurality of the bonding sites of the surface;

wherein the first buffer solution comprises a total concentration of salt or salts of about 100 mM or less.

In some embodiments, the template polynucleotides are single stranded polynucleotides. In other embodiments, the template polynucleotides are double stranded polynucleotides. In some embodiments, the primer oligonucleotides comprise a first type of primer oligonucleotides and a second type of primer oligonucleotides. In further embodiments, the primer oligonucleotides comprise P5 and P7 primers, P15 and P17 primers, PA and PB primers, or PC and PD primers.

In some embodiments, the first plurality bonding sites of the surface comprise or are non-covalent bonding sites. In further embodiments, the non-covalent bonding sites comprise avidin (e.g., streptavidin). In some such embodiments, each of the template polynucleotides comprises or is a biotin moiety that allows for non-covalent bonding with streptavidin.

In other embodiments, the first plurality bonding sites of the surface comprise or are covalent bonding sites. In further embodiments, the covalent bonding sites comprise amino bonding sites, carboxy bonding sites, thiol bonding sites, aldehyde bonding sites, azido bonding sites, hydroxy bonding sites, transcyclooctene bonding sites, norbornene bonding sites, cyclooctyne bonding sites, oxoamine bonding sites, SpyTag bonding sites, Snap-tag bonding sites, CLIP-tag bonding sites, or proteins with N-terminus recognized by sortase, or combinations thereof. In some such embodiments, each of the template polynucleotides comprises a functional moiety that allows for covalent bonding with the covalent bonding sites of the surface. The functional moiety of the template polynucleotides comprises or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other embodiments, the first plurality bonding sites of the surface and the functional moiety of the polynucleotides may be reversed. The covalent bonding between the first plurality bonding sites and the functional moiety of the template polynucleotide include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding site, or sortase-coupling bonding.

In some embodiments, the concentration of the template polynucleotides in the first buffer solution is about 10 pM to about 2000 pM, about 100 pM to about 1000 pM, about 200 pM to about 500 pM, or about 250 pM to about 350 pM. In some embodiments, the first buffer solution has a pH of about 7. In some embodiments, the first buffer solution has a pH of about 3.5 or less. In some embodiments, the first buffer solution further comprises one or more crowding agents. In one embodiment, the crowding agent comprises or is polyethylene glycol (PEG).

In some embodiments, the method described herein further comprises:
contacting a second buffer solution comprising the primer oligonucleotides with the surface of the substrate; and
attaching the primer oligonucleotides to the surface of the substrate by forming covalent bonding or non-covalent bonding between the primer oligonucleotides and the second plurality of the bonding sites of the surface;
wherein the second buffer solution comprises a total concentration of salt or salts of about 250 mM or greater. In one embodiment, the total concentration of salt or salts in the second buffer solution is about 750 mM.

In some embodiments, the primer oligonucleotides comprise the first type of primer oligonucleotides and the second type of primer oligonucleotides. In further embodiments, the primer oligonucleotides comprise P5, P7, P15, P17, PA, PB, PD, or PD primer sequence as described herein. In one embodiment, the primer oligonucleotides comprise P5 primer sequence or P7 primer sequence.

In some embodiments, the second plurality bonding sites of the surface comprise covalent bonding sites. In further embodiments, the second plurality bonding sites of the surface comprise amino bonding sites, carboxy bonding sites, thiol bonding sites, aldehyde bonding sites, azido bonding sites, hydroxy bonding sites, transcyclooctene bonding sites, norbornene bonding sites, cyclooctyne bonding sites, oxoamine bonding sites, SpyTag bonding sites, Snap-tag bonding sites, CLIP-tag bonding sites, or proteins with N-terminus recognized by sortase, or combinations thereof. In some embodiments, each of the plurality of primer oligonucleotides comprises a functional moiety that can form covalent bonding with the second plurality of bonding sites on the surface. The functional moiety of the primer polynucleotides comprises or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other embodiments, the second plurality bonding sites of the surface and the functional moiety of the polynucleotides may be reversed. The covalent bonding between the second plurality bonding sites and the functional moiety of the template polynucleotide include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding site, or sortase-coupling bonding. In one embodiment, the second plurality of bonding sites of the surface comprises azido groups and the functional moiety of the primer oligonucleotide comprises dibenzocyclooctyne (DBCO) moiety, which undergoes strain-promoted copper-free click reaction to form covalent bonding.

In some embodiments, the first plurality of bonding sites and the second plurality of bonding sites are different, which allows for orthogonal reactions with the template polynucleotides and the primer oligonucleotides.

In some embodiments, the method further comprises amplifying the template polynucleotides.

In some embodiments, the surface of the substrate comprises a plurality of patterned nanowells. In some such embodiments, at least about 50%, 60%, 70%, 80%, 90%, 95%, 98%, or 99% of the nanowells is each occupied with at least one cluster of template polynucleotides. In some further embodiments, at least about 50%, 55%, 60%, 65%, 70%, 75%, 80%, 85%, 90%, 95%, 98%, or 99% of the nanowells is each occupied with only one cluster of template polynucleotides, or only one dominant cluster of template polynucleotide.

Additional aspect of the present disclosure relates to a substrate for sequencing, comprising:
template polynucleotides attached to a surface of the substrate through a first plurality of bonding sites via covalent or noncovalent bonding; and
a second plurality of bonding sites for capturing primer oligonucleotides;
wherein the surface of the substrate comprises a plurality of patterned nanowells, and wherein at least about 50%, 55%, 60%, 65%, 70%, 75%, 80%, 85%, 90%, 95%, 98%, or 99% of the nanowells is each occupied with a single template polynucleotide.

### BRIEF DESCRIPTION OF THE DRAWINGS

FIG. 1 illustrates a cross-section view of a standard hybridization based seeding method on a patterned surface of a substrate.

FIG. 2 illustrates a cross-section view of a new hybridization based low salt seeding method on a patterned surface of a substrate, according to an embodiment of the present disclosure.

FIG. 3 illustrates an exemplary workflow of (A) PCR library preparation and (B) PCR-free library preparation.

FIG. 4 illustrates modified library that enable the new hybridization based low salt seeding method using either (A) non-covalent capturing of library DNA strand, or (B) covalent capturing of library DNA strand, according to embodiments of the present disclosure.

### DETAILED DESCRIPTION

The present disclosure relates to compositions and methods for increasing monoclonal clustering during sequencing-by-synthesis (SBS). The methods reverse the standard seeding process by first capturing the library DNA on the solid support in a low salt buffer, and then grafting the primer oligonucleotides (e.g., P5/P7 primers). The low salt seeding condition allows for the electrostatic repulsion of a DNA that is already occupying a given area (i.e., a nanowell) to any additional DNA strands. As a result, any secondary seeding events are disfavored. The process described herein improves the monoclonality of the percent of library strand occupied nanowells. In addition, the methods also improve the occupancy of the nanowells on the solid support, signal intensity, and sequencing data quality, as well as the efficiency of the library capture.

The section headings used herein are for organizational purposes only and are not to be construed as limiting the subject matter described.

### Definition

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, common abbreviations are defined as follows:
dATP Deoxyadenosine triphosphate
dCTP Deoxycytidine triphosphate
dGTP Deoxyguanosine triphosphate
dTTP Deoxythymidine triphosphate
PAZAM Poly(N-(5-azidoacetamidylpentyl) acrylamide-co-acrylamide) of any acrylamide to Azapa ratio
SBS Sequencing-by-synthesis

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “array” refers to a population of different probes (e.g., probe molecules) that are attached to one or more substrates such that the different probes can be differentiated from each other according to relative location. An array can include different probes that are each located at a different addressable location on a substrate. Alternatively or additionally, an array can include separate substrates each bearing a different probe, wherein the different probes can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B 1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached hydrogel refers to a hydrogel that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, the term “reversible covalent bond” refers to a covalent bond that can be cleaved for example under the application of heat, light or other (bio)chemical methods (e.g. by exposure to a degradation agent, such as an enzyme or a catalyst), while a “non-reversible covalent bond” is stable to degradation under such conditions. Non-limiting examples of reversible covalent bonds include thermally or photolytically cleavable cycloadducts (e.g. furan-maleimide cycloadducts), alkenylene linkages, esters, amides, acetals, hemiaminal ethers, aminals, imines, hydrazones, polysulfide linkages (e.g. disulfide linkages), boron-based linkages (e.g. boronic and borinic acids/esters), silicon-based linkages (e.g. silyl ether, siloxane), and phosphorus-based linkages (e.g. phosphite, phosphate) linkages.

As used herein, the term “non-covalent interactions” differs from a covalent bond in that it does not involve the sharing of electrons, but rather involves more dispersed variations of electromagnetic interactions between molecules or within a molecule. Non-covalent interactions can be generally classified into four categories, electrostatic, π-effects, van der Waals forces, and hydrophobic effects. Non-limiting examples of electrostatic interactions include ionic interactions, hydrogen bonding (a specific type of dipole-dipole interaction), halogen bonding, etc. Van der Waals forces are a subset of electrostatic interaction involving permanent or induced dipoles or multipoles. π-effects can be broken down into numerous categories, including (but not limited to) π-π interactions, cation-π & anion-π interactions, and polar-π interactions. In general, π-effects are associated with the interactions of molecules with then-orbitals of a molecular system, such as benzene. The hydrophobic effect is the tendency of nonpolar substances to aggregate in aqueous solution and exclude water molecules. Non-covalent interactions can be both intermolecular and intramolecular. Non-covalent interactions can be both intermolecular and intramolecular.

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

As used herein, the term “ionic bond” refers to a chemical bond between two or more ions that involves an electrostatic attraction between a cation and an anion. For example, the cation may be selected from “metal cations”, as described herein, or “non-metal cations”. Non-metal cations may include ammonium salts (e.g. alkylammonium salts) or phosphonium salts (e.g. alkylphosphonium salts). The anion may be selected from phosphates, thiophosphates, phosphonates, thiophosphonates, phosphinates, thiophosphinates, sulfates, sulfonates, sulfites, sulfinates, carbonates, carboxylates, alkoxides, phenolates and thiophenolates.

As used herein, the term “hydrogen bond” refers to a bonding interaction between a lone pair on an electron-rich atom (e.g. nitrogen, oxygen or fluorine) and a hydrogen atom attached to an electronegative atom (e.g. nitrogen or oxygen).

As used herein, the term “host-guest interaction” refers to two or more groups which are able to form bound complexes via one or more types of non-covalent interactions by molecular recognition, such as ionic bonding, hydrogen bonding, hydrophobic interactions, van der Waals interactions and π-π interactions. For example, the host-guest interaction may include interactions formed between cucubiturils with adamantanes (e.g. 1-adamantylamine), ammonium ions (e.g. amino acids), ferrocenes; cyclodextrins with adamantanes (e.g. 1-adamantylamine), ammonium ions (e.g. amino acids), ferrocenes, calixarenes with adamantanes (e.g. 1-adamantylamine), ammonium ions (e.g. amino acids), ferrocenes; crown ethers (e.g. 18-crown-6, 15-crown-5, 12-crown-4) or cryptands (e.g. [2.2.2]cryptand) with cations (e.g. metal cations, ammonium ions); avidins (e.g. streptavidin) and biotin; and antibodies and haptens.

As used herein, the term “percent passing filter” or “%PF” is a measure of the ability of a nanowell to be successfully ‘read’ during sequencing. As the grafting density increases, there is an initial increase in % PF which is followed by a rapid decline, due to increased polyclonality within a well leading to a reduction in a clean readable target signal. In other words, as the primer density increases, the likelihood of two or more templates hybridizing onto the surface of the well increases. The presence of more than one template increases the likelihood of both templates being amplified leading to polyclonality and an increased likelihood that the signal strength is reduced or not readable. % PF of the occupied wells can therefore be used to measure the degree of clonality. While reference above is made to nanowells, the same concept is applicable to any solid support or substrate.

As used herein, the term “coat,” when used as a verb, is intended to mean providing a layer or covering on a surface. At least a portion of the surface can be provided with a layer or cover. In some cases, the entire surface can be provided with a layer or cover. In alternative cases only a portion of the surface will be provided with a layer or covering. The term “coat,” when used to describe the relationship between a surface and a material, is intended to mean that the material is present as a layer or cover on the surface. The material can seal the surface, for example, preventing contact of liquid or gas with the surface. However, the material need not form a seal. For example, the material can be porous to liquid, gas, or one or more components carried in a liquid or gas. Exemplary materials that can coat a surface include, but are not limited to, a gel, polymer, organic polymer, liquid, metal, a second surface, plastic, silica, or gas.

As used herein the term “analyte” is intended to include any of a variety of analytes that are to be detected, characterized, modified, synthesized, or the like. Exemplary analytes include, but are not limited to, nucleic acids (e.g., DNA, RNA or analogs thereof), proteins, polysaccharides, cells, nuclei, cellular organelles, antibodies, epitopes, receptors, ligands, enzymes (e g kinases, phosphatases or polymerases), peptides, small molecule drug candidates, or the like. An array can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

As used herein the term “contour” is intended to mean a localized variation in the shape of a surface. Exemplary contours include, but are not limited to, wells, pits, channels, posts, pillars, and ridges. Contours can occur as any of a variety of depressions in a surface or projections from a surface. All or part of a contour can serve as a feature in an array. For example, a part of a contour that occurs in a particular plane of a solid support can serve as a feature in that particular plane. In some embodiments, contours are provided in a regular or repeating pattern on a surface.

Where a material is “within” a contour, it is located in the space of the contour. For example, for a well, the material is inside the well, and for a pillar or post, the material covers the contour that extends above the plane of the surface.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. The term can be similarly applied to proteins which are distinguishable as different from each other based on amino acid sequence differences.

As use herein, the term “one cluster of template polynucleotides” refer to a plurality of identical template polynucleotides immobilized on a particular confined location or compartment of a substrate (e.g., within a single nanowell) as a result of amplification of a single template polynucleotide captured at the particular confined location or compartment (e.g., within the same nanowell) of the substrate. The term “one dominant cluster of template polynucleotides” is used in the context of polyclonality as described herein, when clustering result in two or more clusters formed from two or more different template polynucleotides that are seeded in the same confined location or compartment (e.g., within the same nanowell). When an imaging system used in an SBS process may be able distinguish them as separate clusters and the cluster that is responsible for the base calling in sequencing is referred to as “the dominant cluster.”

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “feature” means a location in an array that is configured to attach a particular analyte. For example, a feature can be all or part of a contour on a surface. A feature can contain only a single analyte, or it can contain a population of several analytes, optionally the several analytes can be the same species. In some embodiments, features are present on a solid support prior to attaching an analyte. In other embodiments the feature is created by attachment of an analyte to the solid support.

As used herein, the term “flow cell” is intended to mean a vessel having a chamber where a reaction can be carried out, an inlet for delivering reagents to the chamber and an outlet for removing reagents from the chamber. In some embodiments, the chamber is configured for detection of the reaction that occurs in the chamber (e.g. on a surface that is in fluid contact with the chamber). For example, the chamber can include one or more transparent surfaces allowing optical detection of arrays, optically labeled molecules, or the like in the chamber. Exemplary flow cells include, but are not limited to those used in a nucleic acid sequencing apparatus such as flow cells for the Genome Analyzer®, MiSeq®, NextSeq® or HiSeq® platforms commercialized by Illumina, Inc. (San Diego, Calif.); or for the SOLiD™ or Ion Torrent™ sequencing platform commercialized by Life Technologies (Carlsbad, Calif.). Exemplary flow cells and methods for their manufacture and use are also described, for example, in WO 2014/142841 Al; U.S. Pat. App. Pub. No. 2010/0111768 A1 and U.S. Pat. No. 8,951,781, each of which is incorporated herein by reference.

As used herein, the term “gel material” is intended to mean a semi-rigid material that is permeable to liquids and gases. Typically, a gel material can swell when liquid is taken up and can contract when liquid is removed, e.g., by drying. Exemplary gels include, but are not limited to, those having a colloidal structure, such as agarose; polymer mesh structure, such as gelatin; or cross-linked polymer structure, such as polyacrylamide, silane free acrylamide (see, for example, US Pat. App. Pub. No. 2011/0059865 A1), PAZAM (see, for example, U.S. Pat. No. 9,012,022, which is incorporated herein by reference), and polymers described in U.S. Patent Pub. Nos. 2015/0005447 and 2016/0122816, all of which are incorporated by reference in their entireties. Particularly useful gel material will conform to the shape of a well or other contours where it resides. Some useful gel materials can both (a) conform to the shape of the well or other contours where it resides and (b) have a volume that does not substantially exceed the volume of the well or contours where it resides. In some particular embodiments, the gel material is a polymeric hydrogel.

As used herein, the term “interstitial region” refers to an area in a substrate or on a surface that separates other areas of the substrate or surface. For example, an interstitial region can separate one contour or feature from another contour or feature on the surface. The two regions that are separated from each other can be discrete, lacking contact with each other. In many embodiments the interstitial region is continuous whereas the contours or features are discrete, for example, as is the case for an array of wells in an otherwise continuous surface. The separation provided by an interstitial region can be partial or full separation. Interstitial regions will typically have a surface material that differs from the surface material of the contours or features on the surface. For example, contours of an array can have an amount or concentration of gel material or analytes that exceeds the amount or concentration present at the interstitial regions. In some embodiments the gel material or analytes may not be present at the interstitial regions.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g., found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid, are intended as semantic identifiers for the nucleic acid in the context of a method or composition set forth herein and does not necessarily limit the structure or function of the nucleic acid beyond what is otherwise explicitly indicated. The terms “probe” and “target” can be similarly applied to other analytes such as proteins, small molecules, cells, or the like.

As used herein, the term “surface” is intended to mean an external part or external layer of a solid support or gel material. The surface can be in contact with another material such as a gas, liquid, gel, polymer, organic polymer, second surface of a similar or different material, metal, or coat. The surface, or regions thereof, can be substantially flat or planar. The surface can have surface contours such as wells, pits, channels, ridges, raised regions, pegs, posts or the like.

As used herein, the term “depression” refers to a discrete concave feature in a patterned support having a surface opening that is completely surrounded by interstitial region(s) of the patterned support surface. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

As used herein, the term “substrate” or “solid support” may be used interchangeably and both refer to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (e.g., acrylics, polystyrene and copolymers of styrene and other materials, polypropylene, polyethylene, polybutylene, polyurethanes, Teflon™, cyclic olefins, polyimides, etc.), nylon, ceramics, resins, Zeonor, silica or silica-based materials including silicon and modified silicon, carbon, metals, inorganic glasses, optical fiber bundles, and polymers. A particularly useful material is glass. Other suitable substrate materials may include polymeric materials, plastics, silicon, quartz (fused silica), boro float glass, silica, silica-based materials, carbon, metals including gold, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength, such as one or more of the techniques set forth herein. Conversely, it may be desirable to select a material that does not pass radiation of a certain wavelength (e.g. being opaque, absorptive or reflective). This can be useful for formation of a mask to be used during manufacture of the structured substrate; or to be used for a chemical reaction or analytical detection carried out using the structured substrate. Other properties of a material that can be exploited are inertness or reactivity to certain reagents used in a downstream process; or ease of manipulation or low cost during a manufacturing process manufacture.

Further examples of materials that can be used in the structured substrates or methods of the present disclosure are described in US Pat. App. Pub. No. 2012/0316086 A1 and 2013/0116153, each of which is incorporated herein by reference.

As used herein, the term “well” refers to a discrete contour in a solid support having a surface opening that is completely surrounded by interstitial region(s) of the surface. Wells can have any of a variety of shapes at their opening in a surface including but not limited to round, elliptical, square, polygonal, star shaped (with any number of vertices), etc. The cross section of a well taken orthogonally with the surface can be curved, square, polygonal, hyperbolic, conical, angular, etc. In some embodiments, the well is a microwell or a nanowell.

The P5 and P7 primers are used on the surface of commercial flow cells sold by Illumina Inc. for sequencing on the Specific examples of suitable primers include P5 and/or P7 primers, which are used on the surface of commercial flow cells sold by Illumina, Inc., for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms. The primer sequences are described in U.S. Pat. Pub. No. 2011/0059865 A1, which is incorporated herein by reference. The P5 and P7 primer sequences comprise the following:

Paired end set:
P5: paired end 5′→3′
(SEQ ID NO. 1)
AATGATACGGCGACCACCGAGAUCTACAC
P7: paired end 5′→3′
(SEQ ID NO. 2)
CAAGCAGAAGACGGCATACGAGAT
Single read set:
P5: single read: 5′→3′
(SEQ ID NO. 3)
AATGATACGGCGACCACCGA
P7: single read 5′→3′
(SEQ ID NO. 4)
CAAGCAGAAGACGGCATACGA

In some embodiments, the P5 and P7 primers may comprise a linker or spacer at the 5′ end. Such linker or spacer may be included in order to permit cleavage, or to confer some other desirable property, for example to enable covalent attachment to a polymer or a solid support, or to act as spacers to position the site of cleavage an optimal distance from the solid support. In certain cases, 0-50 spacer, or 10-50 spacer nucleotides may be positioned between the point of attachment of the P5 or P7 primers to a polymer or a solid support. In some embodiments polyT spacers are used, although other nucleotides and combinations thereof can also be used. TET is a dye labeled oligonucleotide having complimentary sequence to the P5/P7 primers. TET can be hybridized to the P5/P7 primers on a surface; the excess TET can be washed away, and the attached dye concentration can be measured by fluorescence detection using a scanning instrument such as a Typhoon Scanner (General Electric). In addition to the P5/P7 primers, other non-limiting examples of the sequencing primer sequences such as P15/P17 primers have also been disclosed in U.S. Publication No. 2019/0352327. In additional, primers PA, PB, PC and PD have been disclosed in U.S. Ser. No. 63/128,663. These additional sequencing primers comprise the following:
P15: 5′→3′

AATGATACGGCGACCACCGAGAT*CTACAC (SEQ. ID. NO. 5), where T* refers to an allyl modified T
P17 primer 5′→3′

YYYCAAGCAGAAGACGGCATACGAGAT (SEQ ID NO. 6), where Y is a diol linker subject to chemical cleavage, for example, by oxidation with a reagent such as periodate, as disclosed in U.S. Publication No. 2012/0309634, which is incorporated by preference in its entirety.

PA: 5′→3′
(SEQ ID NO. 7)
GCTGGCACGTCCGAACGCTTCGTTAATCCGTTGAG
PB: 5′→3′
(SEQ ID NO. 8)
CGTCGTCTGCCATGGCGCTTCGGTGGATATGAACT
PC: 5′→3′
(SEQ ID NO. 9)
ACGGCCGCTAATATCAACGCGTCGAATCCGCAACT
PD: 5′→3′
(SEQ ID NO. 10)
GCCGCGTTACGTTAGCCGGACTATTCGATGCAGC

As used herein, the term “orthogonal” in the context of capturing library template polynucleotides and surface primer oligonucleotides to surface, it is meant that the capture mechanism used to fix the template library to the surface is different from the surface primers used to generate the clusters.

### Methods of Seeding Library DNA

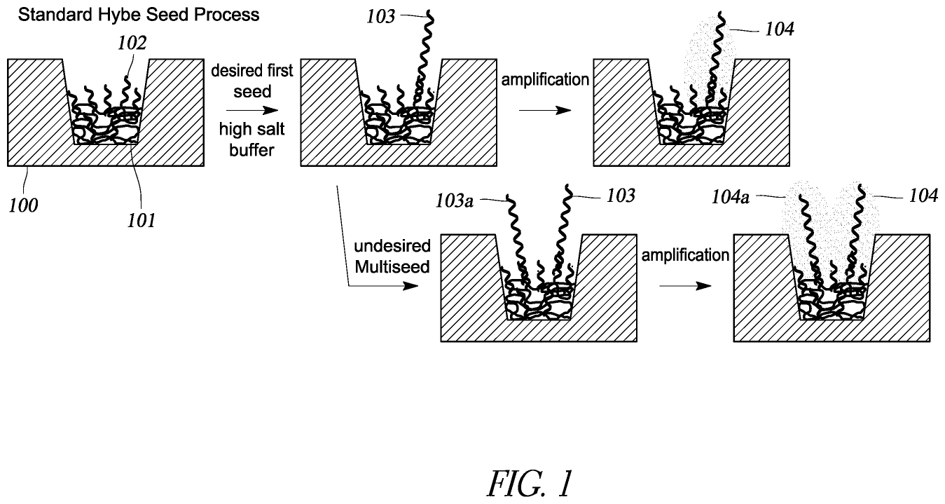

The library seeding methodology that is currently utilized on Illumina's SBS platforms relies on a hybridization event to capture library DNA on the flowcell surface. FIG. 1 illustrates a standard hybridizing based seeding process in a cross-section view of one nanowell 101 on a solid support 100, where the solid support contains a plurality of the nanowells separated by interstitial regions. In this process, nanowell 101 is first functionalized with a hydrogel that allows for the covalent binding with a plurality of primer oligonucleotide 102 to immobilize the primer oligonucleotides to the surface of the solid support. Then, library DNAs (i.e., library strand) are first ligated with adaptors that have sequences which are complementary to the primer oligonucleotides bound on the surface. Then, the library DNAs are flowed over the surface in a buffer solution containing high concentration of salts. Library strand 103 is captured on the surface via hybridization to the surface bound primer oligonucleotide 102. In an ideal process, only one library strand is captured within one single nanowell. After amplification, the monoclonal clustering produces cluster 104. A high salt buffer solution (i.e., the salt concentration is above 100 mM) is needed in order to screen the negatively charged backbone of the library DNA from the negatively charged primer oligonucleotides on the surface. The same screening effect of the high salt buffer enables more than one library strands—library strands 103 and 103a to be co-localized on the flowcell surface within a single nanowell, producing undesirable clusters 104 and 104a in the same nanowell after amplification (polyclonality). The salt concentration as described herein, refers to the concentration of the cations, which are responsible for screening the negatively charged DNA backbone.

Some embodiments of the present disclosure relate to a new process of seeding library DNAs on the surface of the solid support by reversing the library seeding and surface primer grafting steps.

Some aspect of the present disclosure relates to a method of preparing a substrate for sequencing, comprising:
contacting a first buffer solution comprising template polynucleotides with a surface of the substrate, wherein the surface of the substrate comprises a first plurality of bonding sites for capturing template polynucleotides and a second plurality of bonding sites for capturing primer oligonucleotides; and
attaching the template polynucleotides to the surface of the substrate by forming covalent bonding or non-covalent bonding between the template polynucleotides and the first plurality of the bonding sites of the surface;
wherein the first buffer solution comprises a total concentration of salt or salts of about 100 mM or less.

A template polynucleotide as described herein may be of any suitable length, including for sequencing in an SBS process. For example, a template polynucleotide may be about 50 to 2000 nucleotides in length, about 75 to 1000 nucleotides in length, about 100 to 500 nucleotides in length, about 125 to 450 nucleotides in length, about 150 to 400 nucleotides in length, about 175 to 350 nucleotides in length, or about 200 to 300 nucleotides in length.

In some embodiments, the template polynucleotides are single stranded polynucleotides. In other embodiments, the template polynucleotides are double stranded polynucleotides. In some embodiments, the primer oligonucleotides comprise a first type of primer oligonucleotides and a second type of primer oligonucleotides. In further embodiments, the primer oligonucleotides comprise P5 and P7 primers, P15 and P17 primers, PA and PB primers, or PC and PD primers. In one embodiment, the primer oligonucleotides P5 and P7 primers described herein. The primer oligonucleotides are also referred to as surface primer or clustering primer because they are grafted on the surface of the solid support in order to carry out amplification of the seeded template polynucleotides and forming clusters.

In some embodiments of the seeding method described herein, the first buffer solution comprises about 90, 85, 80, 75, 70, 65, 60, 55, 50, 45, 40, 35, 30, 25, 20, 15, 10, or 5 mM salt(s) or less. The first buffer solution may comprise one or more buffering agents and one or more non-buffering salts. None limiting examples of buffering agents include Tris, glycine, sodium ascorbate, sodium phosphate, HEPES, MOPS, PIPES, TAPS, etc. Non-limiting example of non-buffering salts include KCl, NaCl, LiCl, MgCl₂, MnCl₂, etc. In some embodiments, the total amount of salt concentration refers to the total concentration of both the buffering agents and the non-buffering salt cations in the first buffer solution. In other embodiments, the total amount of salt concentration refers to the total concentration of the non-buffering salt cations in the first buffer solution. In still other embodiments, the total amount of salt concentration refers to the total concentration of the inorganic salt cations in the first buffer solution. In further embodiments, the first buffer solution may also comprise one or more surfactants, such as Tween-20 or sodium dodecyl sulfate (SDS). In some embodiments, the first buffer solution further comprises one or more crowding agents. In one embodiment, the crowding agent comprises or is polyethylene glycol (PEG).

In some embodiments, the pH of the first buffer solution may range from about 3 to about 11. In some embodiments, the first buffer solution has a pH of about 7. In some embodiments, the first buffer solution has a pH of about 3.5 or less. In some instance, it has been observed that the negative surface charge that is inherent on many of the surfaces (e.g., glass surface or resin surface) tend to repel DNA. This effect may be mitigated by seeding at lower pH where surface charges are reduced by protonation (phosphate remains unprotonated at pH greater than about 3.5). In addition, surface modifications may be implemented to reduce the negative charge of the substrate surface in order to improve the low salt seeding method described herein.

In some embodiments of the seeding method described herein, the first plurality bonding sites of the surface comprise or are non-covalent bonding sites. In further embodiments, the non-covalent bonding sites comprise avidin (e.g., streptavidin). In some such embodiments, each of the template polynucleotides comprises or is a biotin moiety that allows for non-covalent bonding with streptavidin. In other embodiments, the first plurality bonding sites of the surface comprise or are covalent bonding sites. In further embodiments, the covalent bonding sites comprise amino bonding sites, carboxy bonding sites, thiol bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxoamine bonding sites, SpyTag bonding sites, Snap-tag bonding sites, CLIP-tag bonding sites, or proteins with N-terminus recognized by sortase, or combinations thereof. In some such embodiments, each of the template polynucleotides comprises a functional moiety that allows for covalent bonding with the covalent bonding sites of the surface. The functional moiety of the template polynucleotides comprises or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other embodiments, the first plurality bonding sites of the surface and the functional moiety of the polynucleotides may be reversed. The covalent bonding between the first plurality bonding sites and the functional moiety of the template polynucleotide include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding site, or sortase-coupling bonding. As described herein, each of the moiety at the surface bonding sites or the functional moiety of the template polynucleotide may be either unsubstituted or substituted.

A non-exclusive list of complementary binding partners is presented in Table 1:

| | Exemplary fist bonding site on | Exemplary functional moiety on |
|---|---|---|
| | surface of substrate or functional | template polynucleotide or first |
| Bonding site | moiety on template polynucleotide | bonding site on surface of substrate |
| amine-NHS | amine group, -NH₂ | 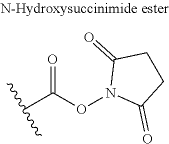 [SMILES: *C(=O)ON1C(=O)CCC1=O] |
| amine- imidoester | amine group, -NH₂ | 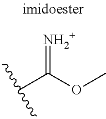 [SMILES: *C(=[NH2+])OC] |
| amine- pentofluorophenyl ester | amine group, -NH₂ | 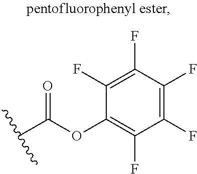 [SMILES: *C(=O)Oc1c(F)c(F)c(F)c(F)c1F] |
| amine- hydroxymethyl phosphine | amine group, -NH₂ | 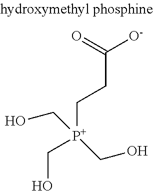 [SMILES: O=C([O-])CC[P+](CO)(CO)CO] |
| amine- carboxylic acid | amine group, -NH₂ | carboxylic acid group, -C(═O)OH (e.g., following activation of the carboxylic acid by a carbodiimide such as EDC (1- ethyl-3-(-3-dimethylaminopropyl) carbodiimide hydrochloride) or DCC (N′,N′-dicyclohexyl carbodiimide) to allow for formation of an amide bond of the activated carboxylic acid with an amine group) |
| thiol- maleimide | thiol, -SH | 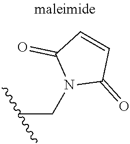 [SMILES: *CN1C(=O)C=CC1=O] |
| thiol- haloacetyl | thiol, -SH | 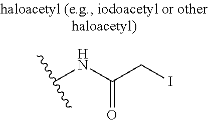 [SMILES: *NC(=O)CI] |
| thiol-pyridyl disulfide | thiol, -SH | 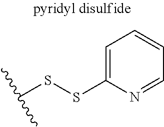 [SMILES: *SSc1ccccn1] |
| thiol- thiosulfonate | thiol, -SH | 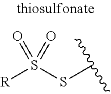 |
| thiol-vinyl sulfone | thiol, -SH | 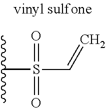 [SMILES: *S(=O)(=O)C=C] |
| aldehyde- hydrazide | aldehyde, -C(═O)H | 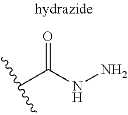 [SMILES: *C(=O)NN] |
| aldehyde- alkoxyamine | aldehyde, -C(═O)H | 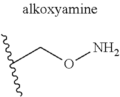 [SMILES: *CON] |
| hydroxy- isocyanate | hydroxyl, -OH | 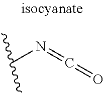 [SMILES: *N=C=O] |
| azide-alkyne | azide, -N₃ | 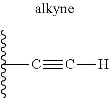 [SMILES: *C#C] |
| azide- phosphine | azide, -N₃ | 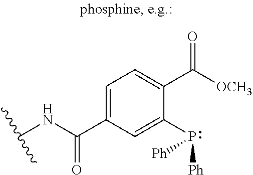 |
| azide- cyclooctyne | azide, -N₃ | 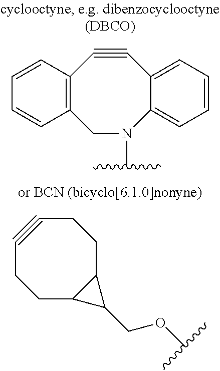 [SMILES: *N1Cc2ccccc2C#Cc2ccccc21 ; *OCC1C2CCC#CCCC21] |
| azide- norbornene | azine, -N₃ | 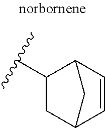 [SMILES: *C1CC2C=CC1C2] |
| transcyclooctene- tetrazine | 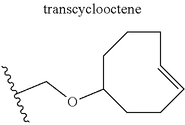 [SMILES: *COC1CC/C=C/CCC1] | 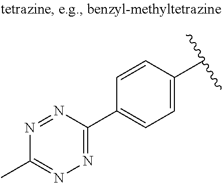 [SMILES: *c1ccc(-c2nnc(C)nn2)cc1] |
| norbornene- tetrazine | 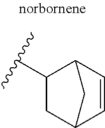 [SMILES: *C1CC2C=CC1C2] | 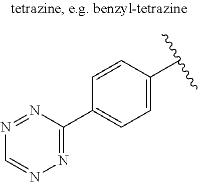 [SMILES: *c1ccc(-c2nncnn2)cc1] |
| oxime | aldehyde or ketone (e.g., amine group or N-terminus of polypeptide converted to an aldehyde or ketone by pyroxidal phosphate) | alkoxyamine |
| SpyTag- SpyCatcher | SpyTag: amino acid sequence AHIVMVDAYKPTK | SpyCatcher amino acid sequence: MKGSSHHHHHHVDIPTTENLYFQ GAMVDTLSGLSSEQGQSGDMTIEE DSATHIKFSKRDEDGKELAGATME LRDSSGKTISTWISDGQVKDFYLY PGKYTFVETAAPDGYEVATAITFT VNEQGQVTVNGKATK |
| SNAP-tag-O⁶- Benzylguanine | SNAP-tag (O-6-methylguanine- DNA methyltransferase) | 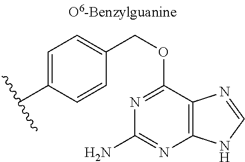 [SMILES: *c1ccc(COc2nc(N)nc3[nH]cnc23)cc1] |
| CLIP-tag-O²- benzylcytosine | CLIP-tag (modified O-6- methylguanine-DNA methyltransferase) | 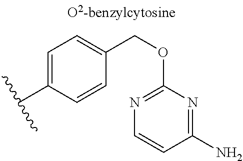 [SMILES: *c1ccc(COc2nccc(N)n2)cc1] |
| Sortase- coupling | -Leu-Pro-X-Thr-Gly | -Gly₍₃₋₅₎ |

In some embodiments, the concentration of the template polynucleotides in the first buffer solution is about 10 pM to about 2000 pM, about 100 pM to about 1000 pM, about 200 pM to about 500 pM, or about 250 pM to about 350 pM. In one embodiment, the concentration of the template polynucleotides in the first buffer solution is about 250 pM.

Surface Primers Grafting

In some embodiments, the method described herein further comprises:

contacting a second buffer solution comprising the primer oligonucleotides with the surface of the substrate; and attaching the primer oligonucleotides to the surface of the substrate by forming covalent bonding or non-covalent bonding between the primer oligonucleotides and the second plurality of the bonding sites of the surface;

wherein the second buffer solution comprises a total concentration of salt or salts of about 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, or 1000 mM, or greater. In one embodiment, the total concentration of salt or salts in the second buffer solution is about 750 mM.

In some embodiments, the primer oligonucleotides comprise the first type of primer oligonucleotides and the second type of primer oligonucleotides. In further embodiments, the primer oligonucleotides comprise P5, P7, P15, P17, PA, PB, PD, or PD primer sequence as described herein. In one embodiment, the primer oligonucleotides comprise P5 primer sequence or P7 primer sequence.

In some embodiments, the second plurality bonding sites of the surface comprises comprise covalent bonding sites. In further embodiments, the second plurality bonding sites of the surface comprise amino bonding sites, carboxy bonding sites, thiol bonding sites, aldehyde bonding sites, azido bonding sites, hydroxy bonding sites, transcyclooctene bonding sites, norbornene bonding sites, cyclooctyne bonding sites, oxoamine bonding sites, SpyTag bonding sites, Snap-tag bonding sites, CLIP-tag bonding sites, or proteins with N-terminus recognized by sortase, or combinations thereof. In some embodiments, each of the plurality of primer oligonucleotides comprises a functional moiety that can form covalent bonding with the second plurality of bonding sites on the surface. The functional moiety of the primer polynucleotides comprises or is selected from a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other embodiments, the second plurality bonding sites of the surface and the functional moiety of the polynucleotides may be reversed. The covalent bonding between the second plurality bonding sites and the functional moiety of the template polynucleotide include but not limited to amine-NHS ester bonding, amine-imidoester bonding, amine-pentofluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxoamine-aldehyde bonding, SpyTag-SpyCatcher bonding, Snap-tag-O⁶-benzylguanine bonding, CLIP-tag-O²-benzylcytosine bonding site, or sortase-coupling bonding. In one embodiment, the second plurality of bonding sites of the surface comprises azido groups and the functional moiety of the primer oligonucleotide comprises dibenzocyclooctyne (DBCO) moiety, which undergoes strain-promoted copper-free click reaction to form covalent bonding. Additional exemplary embodiments of the complementary partners (between the second plurality of bonding sites and the functional moiety of the surface primer oligonucleotides are presented in Table 1 above.

In some embodiments, either the first or the second plurality of bonding sites may be attached to the surface of the substrate through a polymer (including copolymer, may be random, block, linear, and/or branched copolymers) or a hydrogel, each of which comprising two or more recurring monomer units in any order or configuration, and may be linear, cross-linked, or branched, or a combination thereof. In an example, the polymer may be a heteropolymer and the heteropolymer may include an acrylamide monomer, such as

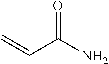

or a substituted analog thereof (“substituted” referring to the replacement of one or more hydrogen atoms in a specified group with another atom or group). In an example, the polymer is a heteropolymer and may further include an azido-containing acrylamide monomer. The polymer or hydrogel may be coated on the surface either by covalent or non-covalent attachment.

In some embodiments, the heteropolymer includes:

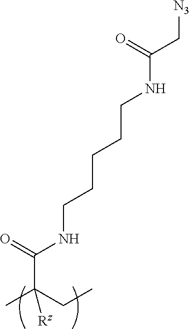

and optionally

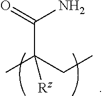

where each R^{z} is independently H or C₁₋₄ alkyl. In an example, a polymer used may include examples such as a poly(N-(5-azidoacetamidylpentyl)acrylamide-co-acrylamide), also known as PAZAM:

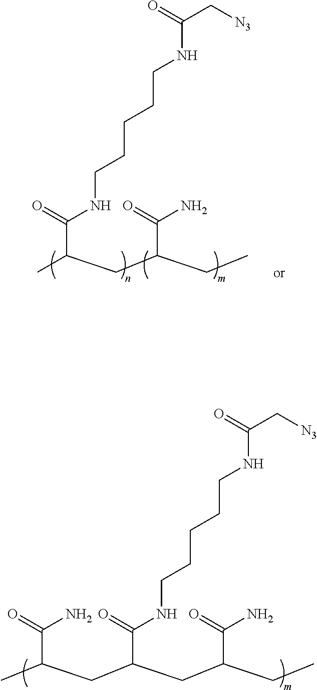

wherein n is an integer in the range of 1-20,000, and m is an integer in the range of 1-100,000. In some examples, the acrylamide monomer may include an azido acetamido pentyl acrylamide monomer:

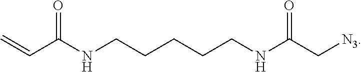 [SMILES: C=CC(=O)NCCCCCNC(=O)CN=[N+]=[N-]]

In some examples, the acrylamide monomer may include an N-isopropylacrylamide

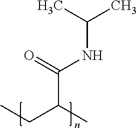

In some embodiments, the heteropolymer may include the structure:

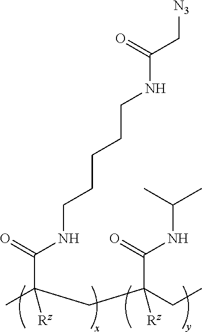

wherein x is an integer in the range of 1-20,000, and y is an integer in

the range of 1-100,000, or

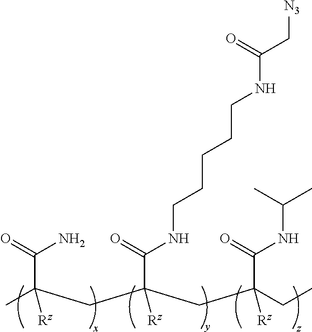

wherein y is an integer in the range of 1-20,000 and x and z are integers wherein the sum of x and z may be within a range of from 1 to 100,000, where each RZ is independently H or C₁₋₄ alkyl and a ratio of x:y may be from approximately 10:90 to approximately 1:99, or may be approximately 5:95, or a ratio of (x:y):z may be from approximately 85:15 to approximately 95:5, or may be approximately 90:10 (wherein a ratio of x:(y:z) may be from approximately 1:(99) to approximately 10:(90), or may be approximately 5:(95)), respectively.

In any embodiments of the method described herein, the first plurality bonding sites on the solid support and/or the functional moiety of the template polynucleotides may comprise a functional group selected from substituted or unsubstituted alkenyl, substituted or unsubstituted alkynyl, substituted or unsubstituted cycloalkenyl (e.g. norbornenyl, cis- or trans-cyclooctenyl), substituted or unsubstituted cycloalkynyl (e.g. cyclooctynyl, dibenzocyclooctynyl, bicyclononynyl), azido, substituted or unsubstituted tetrazinyl, substituted or unsubstituted hydrazonyl, substituted or unsubstituted tetrazolyl, aldehydes, ketones, carboxylic acids, sulfonyl fluorides, diazo (e.g. α-diazocarbonyl), substituted or unsubstituted oximes, hydroximoyl halides, nitrile oxide, nitrone, substituted or unsubstituted amino, substituted or unsubstituted hydrazines, thiol, or hydroxyl.

In any embodiments of the method described herein, the second plurality bonding sites on the solid support and/or the functional moiety of the surface primer oligonucleotides may comprise a functional group selected from substituted or unsubstituted alkenyl, substituted or unsubstituted alkynyl, substituted or unsubstituted cycloalkenyl (e.g. norbornenyl, cis- or trans-cyclooctenyl), substituted or unsubstituted cycloalkynyl (e.g. cyclooctynyl, dibenzocyclooctynyl, bicyclononynyl), azido, substituted or unsubstituted tetrazinyl, substituted or unsubstituted hydrazonyl, substituted or unsubstituted tetrazolyl, aldehydes, ketones, carboxylic acids, sulfonyl fluorides, diazo (e.g. α-diazocarbonyl), substituted or unsubstituted oximes, hydroximoyl halides, nitrile oxide, nitrone, substituted or unsubstituted amino, substituted or unsubstituted hydrazines, thiol, or hydroxyl.

In some embodiments, the first plurality of bonding sites and the second plurality of bonding sites are different. In further embodiments, the functional moiety of the template polynucleotides is different from the functional moiety of the surface primer oligonucleotides. In further embodiments, the chemistry for capturing the template polynucleotides are orthogonal to the chemistry for capturing the surface primers.

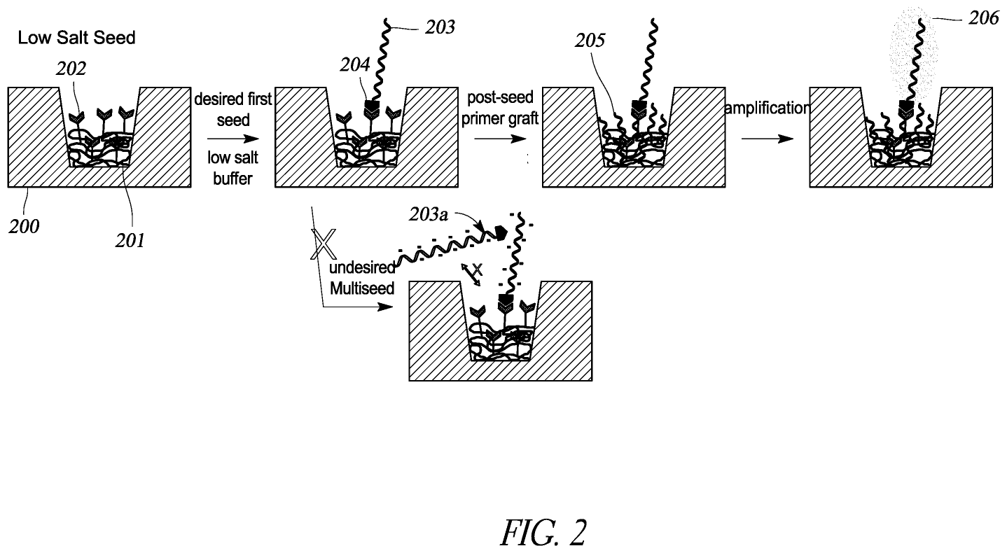

An embodiment of the method described herein is illustrated in FIG. 2. FIG. 2 illustrates a cross-section view of one nanowell 201 on a solid support 200, where the solid support contains a plurality of the nanowells separated by interstitial regions. In the first step, nanowell 201 is first functionalized with a first plurality of binding sites 202 that allows for capturing library DNAs (either through covalent capturing or non-covalent capturing chemistries). Then, library DNAs (i.e., library strand) are first ligated with adaptors that have sequences which are complementary to the primer oligonucleotides, then modified with functional group 204 that can be captured by the first plurality of binding sites on the surface. Then, the library DNAs are flowed over the surface in a buffer solution containing low concentration of salts. Library strand 203 is captured on the surface via interaction between the first capture side 204 and the functional group 204. In this process, it is possible to avoid co-localization of multiple library strands within a single nanowell on the flowcell surface, since secondary seeding events (such as the introduction of a second library strand 203a) are disfavored due to electrostatic repulsion from library strand that is already occupying a nanowell, without the screening of a high salt buffer. After seeding, the primer oligonucleotides 205 (such as the P5 or P7 primer) are then grafted on the surface. In some embodiments, the primer oligonucleotides may be grafted on the surface in a high salt buffer. In other embodiments, the primer oligonucleotides may be grafted on the surface in a low salt buffer or even pure water. The subsequent amplification step produces the monoclonal cluster 206 on the surface.

In some embodiments of the low salt seeding method described herein, the method improves % PF to at least 60%, 65%, 70%, 75%, 80%, 85%, 90%, 95%, 98%, or 99%. In some further embodiments, the method improves monoclonality such that at least 50%, 55%, 60%, 65%, 70%, 75%, 80%, 85%, 90%, 95%, 98%, or 99% of the occupied nanowells on the surface of the substrate present sufficient monoclonality to produce SBS data of adequate quality (i.e., there is only one cluster of template polynucleotides or only one dominant cluster of template polynucleotides). In further embodiments, the method improves the overall occupancy of the nanowells on the surface of the substrate such that at least 60%, 65%, 70%, 75%, 80%, 85%, 90%, 95%, 98% or 99% of the available nanowells on the surface are occupied with template polynucleotides. The nanowells may also be in any other form of depressions or contours on the surface, in any shape or dimension as described herein. In further embodiments, the nanowells, depressions or contours on the surfaces form patterned arrays, where the nanowells, depressions or contours are separated by interstitial regions.

Library Preparation

Library preparation is the first step in any high-throughput sequencing platform. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adapters (adapter sequences). This may be followed by amplification and sequencing. The original sample DNA fragments are referred to as “inserts.” Alternatively, “tagmentation” can be used to attach the sample DNA to the adapters. In tagmentation, double-stranded DNA is simultaneously fragmented and tagged with adapter sequences and PCR primer binding sites. The combined reaction eliminates the need for a separate mechanical shearing step during library preparation. The target polynucleotides may advantageously also be size fractionated prior to modification with the adaptor sequences.

As used herein, an “adapter” sequence comprises a short sequence-specific oligonucleotide that is ligated to the 5′ and 3′ ends of each DNA (or RNA) fragment in a sequencing library as part of library preparation.

As will be understood by the skilled person, a double-stranded nucleic acid will typically be formed from two complementary polynucleotide strands comprised of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridized to a complementary strand—for example, a long polynucleotide strand hybridized to a short nucleotide primer—it may still be referred to herein as a single stranded nucleic acid.

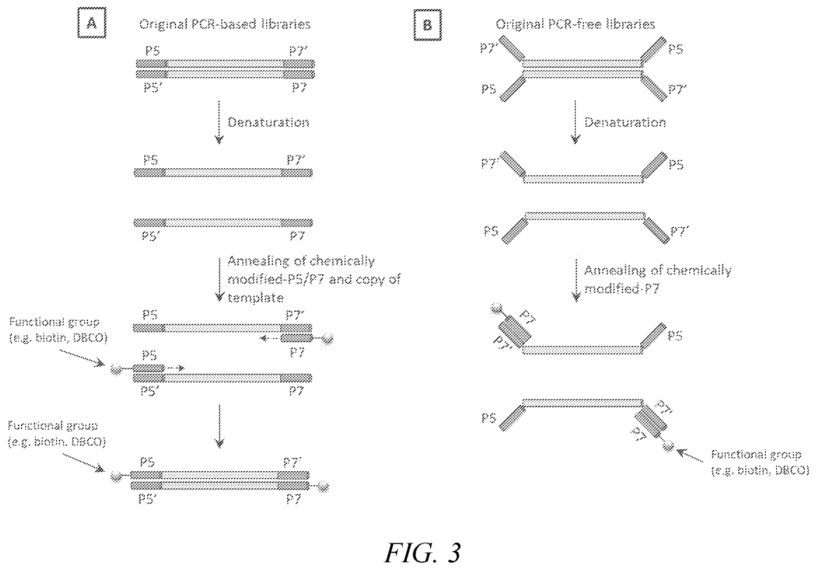

In some embodiments of the method described herein, library DNA strands are modified to possess a functional group capable of being captured at the surface of the substrate, for example, being captured by the first plurality of bonding sites on the surface of the substrate as described herein. FIG. 3 illustrates two different workflows for preparing modified DNA library: (A) PCR-based libraries and (B) PCR-free libraries. In some embodiments, the chemical functionalization of the library DNA can be incorporated covalently through a round of PCR amplification prior to clustering (PCR-based libraries) or added through modification to existing workflow steps including adapter hybridization during library preparation (PCR-free libraries).

For PCR-based library preparation, a double-stranded template is prepared, comprising fragmenting the library and ligating the adaptor sequence to the insert. This results in an insert sequence flanked at its 5′ and 3′ ends by adaptor sequences comprising primer-binding sequences. Once the library is formed, the library is denatured, and the desired chemical functionalization is introduced during PCR enrichment. As shown in FIG. 3(A), the complement of the primer-binding sequence anneals to its complement (e.g. P5′ or P7′) in the template strand. Extension of the P7 or P5 primer leads to a double-stranded template with, for example, biotin or BDCO moiety present at the 5′ ends.

A different workflow is applied to PCR-free library preparation. In FIG. 3(B), a PCR-free library is constructed by standard procedures and then denatured to produce free single stranded libraries. Upon neutralization of the denaturation reaction, a blocking oligo bearing the desired chemical functionalization is added in excess. This oligo contains, for example, biotin or DBCO, where the sequence is complementary to P7′ on the PCR-free 3′ termini. These blocking oligos affectively render P7′ double stranded so it cannot anneal to the FC, while at the same time providing a functionalization available for chemical binding of the library in the nanowell.

In addition to the sequences that are complementary to the surface primers (such as P5′ or P7′), additional sequences may be added to the library strands. The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO 2005/068656, whose contents are incorporated herein by reference in their entirety. The tag can be read at the end of the first read by hybridizing an index read primer, or at the end of the second read, by using the surface primers as index read primers P7. The invention is not limited by the number of reads per cluster, for example two reads per cluster: three or more reads per cluster are obtainable simply by rehybridizing a first extended sequencing primer, and rehybridizing a second primer before or after a cluster repopulation/strand resynthesis step. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridizes) to a portion of the sequencing binding site on the template strand. The DNA polymerase enzyme binds to this site and incorporates complementary nucleotides base by base into the growing opposite strand. In one embodiment, the sequencing process comprises a first and second sequencing read. The first sequencing read may comprise the binding of a first sequencing primer (read 1 sequencing primer) to the first sequencing binding site (e.g. SB S3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may comprise binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

Once a double stranded nucleic acid template library is formed, typically, the library will be subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one embodiment, chemical denaturation, such as NaOH or formamide, is used. In another embodiment, the DNA is thermally denatured by heating.

Following denaturation, a single-stranded template library can be contacted in free solution onto a solid support comprising surface capture moieties (for example P5 and P7 primers). This solid support is typically a flowcell, although in alternative embodiments, seeding and clustering can be conducted off-flowcell using, for example, microbeads or the like.

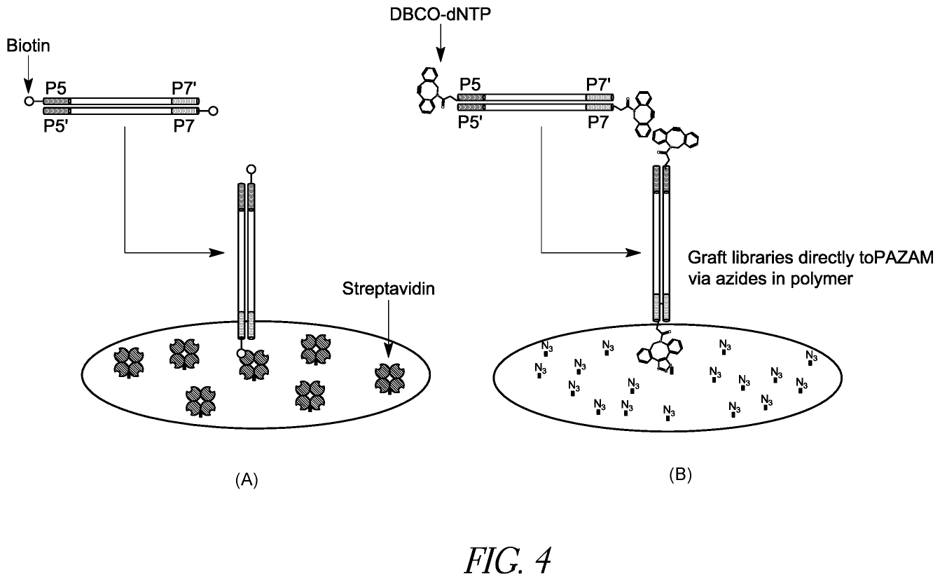

FIG. 4 illustrates two examples of modified library that enable the new hybridization based low salt seeding method using either (A) non-covalent capturing of library DNA strand (template polynucleotide), or (B) covalent capturing of library DNA strand, according to embodiments of the present disclosure.

In one embodiment as illustrated in FIG. 4(A), a double stranded template polynucleotide with biotin functionality at the 5′ ends of the P5 or P7 adaptor sequence is prepared from a workflow described in FIG. 3(A). The surface of the substrate comprises a plurality of avidin bonding sites (e.g., streptavidin), which allows for the non-covalent interaction between streptavidin and biotin moiety, resulting in the capture of the template polynucleotides. Alternatively, the solid support may comprise biotin and the template polynucleotide may be functionalized with an avidin moiety. Other non-covalent interaction may also be used. These non-covalent interactions may include one or more of ionic bonds, hydrogen bonds, hydrophobic interactions, π-π interactions, van der Waals interactions and host-guest interactions described herein. Where non-covalent interactions are used, the type of interaction is not particularly limited, provided that the interactions are (collectively) sufficiently strong for the template to remain attached to the solid support during extension. The non-covalent interactions may also be weak enough such that the template can then be removed from the solid support once a copy of the template has been extended on a surface primer.

In another embodiment as illustrated in FIG. 4(B), the template polynucleotide may be attachable to the solid support by covalent bonds. The surface of the substate comprises a plurality of azido bonding site (e.g., the azido bonding sites are introduced a PAZAM coated surface). The

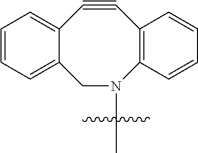 [SMILES: *N1Cc2ccccc2C#Cc2ccccc21]

double stranded template polynucleotide with DBCO functionality at the 5′ ends of the P5 or P7 adaptor sequence is prepared from a workflow described in FIG. 3(A). The template polynucleotide is covalently bounded to the surface by reaction of the DBCO and azido groups,

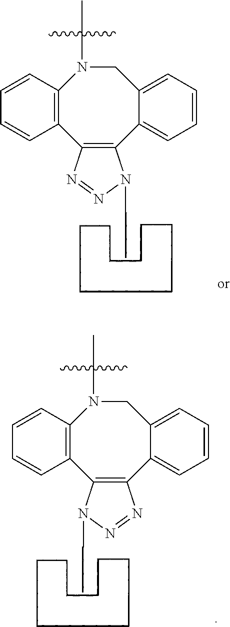 [SMILES: *N1Cc2ccccc2-c2c(nnn2C)-c2ccccc21 ; *N1Cc2ccccc2-c2nnn(C)c2-c2ccccc21]

forming Where covalent bonds are used, the bond may be stable such that the template remains attached to the solid support. Non-limiting examples of covalent bonds include alkylene linkages, alkenylene linkages, alkynylene linkages, ether linkages (e.g. ethylene glycol, propylene glycol, polyethylene glycol), amine linkages, ester linkages, amide linkages, carbocyclic or heterocyclic linkages, sulfur-based linkages (e.g. thioether, disulfide, polysulfide, or sulfoxide linkages), acetals, hemiaminal ethers, aminals, imines, hydrazones, boron-based linkages (e.g. boronic and borinic acids/esters), silicon-based linkages (e.g. silyl ether, siloxane), and phosphorus-based linkages (e.g. phosphite, phosphate).

In some embodiments, the covalent bond may be a reversible covalent bond such that the template can then be removed from the solid support once a copy of the template has been extended on a surface primer. In other embodiments, the covalent bond may be a non-reversible bond.

Any suitable bioconjugation methods for adding functional moiety to the template polynucleotides or surface primers may be used. Modified nucleotides may be commercially available possessing the functional moieties or structures, and methods for attaching or including them to polymer, a nucleotide, or polynucleotide are also known. Bifunctional linker molecules with a moiety or structure from one complementary pair of bonding partners listed in Table 1 at one end and a moiety or structure from another complementary pair of bonding partners listed in Table 1 may also be commercially available. The template polynucleotide or the primer oligonucleotides may be bound to one end of such a linker, resulting in the initial moiety or structure being effectively replaced with another, i.e., the moiety or structure present on the other end of the linker.

For example, a bifunctional linker may have on one end a moiety from among those listed in Table 1, such as an NHS-ester group. At the other end it may have another group, such as an azido group. The ends may be connected to each other by a linker, such as, for example, one or more PEG groups, alkyl chain, combinations thereof in a linking sequence, etc. If a template polynucleotide has an amine group, the NHS-ester end of the bifunctional linker can be bound to the amine group, leaving the free azido end available for bonding to the first plurality of bonding sites bearing a bonding partner for an azido group (e.g., alkyne, phosphine, cyclooctyne, or norbornene, etc.). Many other examples of bifunctional linkers are commercially available including on an end a moiety identified in Table 1 for forming one type of bonding site or functional moiety and on the other end a different moiety identified in Table 1 for forming another type of bonding site or functional moiety.

In another example, the template polynucleotide may include a first polypeptide sequence, and the first plurality of the bonding sites of the substrate may have a second polypeptide sequence capable of covalently bonding to the first polypeptide sequence of the template polynucleotide. Non-limiting examples of such pairs include the SpyTag/SpyCatcher system, the Snap-tag/O⁶-Benzylguanine system, and the CLIP-tag/O²-benzylcytosine system. Similarly, the surface primer oligonucleotides and the second plurality of the bonding sites of the substrate may have the first polynucleotide sequence and the second polynucleotide sequence. Amino acid sequences for the complementary pairs of the SpyTag/SpyCatcher system and polynucleotides encoding them may be available. Examples of sequences are provided in Table 1. Several amino acid site mutations for a SpyTag sequence and for a SpyCatcher sequence may be available for inclusion in recombinant polypeptides. A Snap-tag is a functional O⁶-methylguanine-DNA methyltransferase, and a CLIP-tag is a modified version of Snap-tag. Nucleotide sequences encoding Snap-tag, CLIP-tag, SpyCatcher, may be commercially available for subcloning and inclusion in engineered polypeptide sequences.

Alternatively, complementary pairs for covalent attachment of the template polynucleotides or surface primers on the first or second plurality of bonding sites respectively may be covalently attached to each other via an enzymatically catalyzed formation of a covalent bond. For example, a template polynucleotide and a first bonding site may include motifs capable of covalent attachment to each other by sortase-mediated coupling, e.g. a LPXTG amino acid sequence on one and an oligo glycine nucleophilic sequence on the other (with a repeat of, e.g., from 3 to 5 glycines). Sortase-mediated transpeptidation may then be carried out to result in covalent attachment of the scaffold and template polynucleotide at the single template site.

In another example, the template polynucleotides (or surface primers), the first plurality of bonding sites (or second plurality of bonding sites) may include or be attached to complementary peptide binding sites. For example, the template polynucleotide may include or be attached to peptide sequences that may bind to each other as complementary pairs of a coiled coil motif. A coiled coil motif is a structural feature of some polypeptides where two or more polypeptide strands each form an alpha-helix secondary structure and the alpha-helices coil together to form a tight non-covalent bond. A coiled coil sequence may include a heptad repeat, a repeating pattern of the seven amino acids HPPHCPC (where H indicates a hydrophobic amino acid, C typically represents a charged amino acid and P represents a polar, hydrophilic amino acid). An example of a heptad repeat is found in a leucine zipper coiled coil, in which the fourth amino acid of the heptad is frequently leucine.

In another example, the template polynucleotides (or surface primers), the first plurality of bonding sites (or second plurality of bonding sites) may include or be attached to peptide pairs that bind together non-covalently. An example includes a biotin-avidin binding pair. Biotin and avidin peptides (such as avidin, streptavidin, and neutravidin, all of which are referred to collectively as “avidin” herein unless specifically stated otherwise) form strong noncovalent bonds to each other. One part of such pair, whether binding portion of biotin or of avidin, may be part of or attached to either the template polynucleotides or the surface primers, with the complementary part correspondingly part of or attached to the first plurality of bonding sites or second plurality of bonding sites, or vice versa, permitting non-covalent attachment therebetween.

Numerous methods are available for including one or more biotin moiety in or adding one or more biotin moiety to a DNA molecule or template polynucleotide. For example, biotinylated nucleotides are commercially available for incorporation into a DNA molecule by a polymerase, and kits are commercially available for adding a biotin moiety to a polynucleotide or a polypeptide. Biotin residues can also be added to amino acids or modified amino acids or nucleotides or modified nucleotides. Linking chemistries shown in Table 1 can also be used for adding a biotin group to proteins such as on carboxylic acid groups, amine groups, or thiol groups. Several biotin ligase enzymes are also available for enzymatically targeted biotinylation such as of polypeptides (e.g., of the lysine reside of the AviTag amino acid sequence GLNDIFEAQKIEWHE included in a polypeptide). A genetically engineered ascorbate peroxidase (APEX) is also available for modifying biotin to permit biotinylation of electron-rich amino acids such as tyrosine, and possibly tryptophan, cysteine, or histidine.

In another example, a polypeptide including the amino acid sequence DSLEFIASKLA may be biotinylated (at the more N-terminal of the two S residues present in the sequence), which is a substrate for Sfp phosphopantetheinyl transferase-catalyzed covalent attachment thereto with small molecules conjugated to coenzyme A (CoA). For example, a polypeptide including this sequence could be biotinylated through covalent attachment thereto by a CoA-biotin conjugate. This system may also be used for attaching many other types of bonding moieties or structures identified in Table 1 for use in creating bonding sites for a scaffold to bond to a DNA molecule or polypeptide or other molecule as disclosed herein. For example, a CoA conjugated to any of the reactive pair moieties identified in Table 1 could be covalently attached to a polypeptide containing the above-identified sequence by Sfp phosphopantetheinyl transferase, thereby permitting bonding of another composition thereto that includes the complementary bonding partner.

Other enzymes may be used for adding bonding moiety to a polypeptide. For example, a lipoic acid ligase enzyme can add a lipoic acid molecule, or a modified lipoic acid molecule including a bonding moiety identified in Table 1 such as an alkyne or azide group, can be covalently linked to the amine of a side group of a lysine reside in an amino acid sequence DEVLVEIETDKAVLEVPGGEEE or GFEIDKVWYDLDA included in a polypeptide. In another example, a scaffold, template polynucleotide, or other polypeptide or DNA molecule included therein or intended to be bonded thereto may include or be attached to an active serine hydrolase enzyme. Fluorophosphonate molecules become covalently linked to serine residues in the active site of serine hydrolase enzymes. Commercially available analogs of fluorophosphonate molecules including bonding moieties identified in Table 1, such as an azide group or a desthiobiotin group (an analog of biotin that can bind to avidin). Thus, such groups can be covalently attached to serine hydrolase enzyme included in or attached to a polypeptide or DNA molecule used in or attached to a scaffold as disclosed herein and such bonding moiety or structure can be covalently added thereto by use by attachment of a suitable modified fluorophosphonate molecule for creating a bonding site on such protein for a complementary bonding partner from Table 1 (such as for azide-alkyne, azide-phosphine, azide-cyclooctyne, azide-norbornene, or desthiobiotin-avidin bonding).

Any of the foregoing methods of biotinylating compositions to promote bonding to a polypeptide including an avidin sequence (such as an avidin polypeptide included in or attached to another composition), or otherwise adding functional groups to polypeptides, as part of a scaffold, attached to a scaffold, part of an accessory, or attached to an accessory or template polynucleotide, for bonding between a scaffold and a template polynucleotide or between a scaffold and an accessory, may be used for permitting or promoting bonding between such components as disclosed herein.

### Amplification

By way of brief example, following attachment of the P5 and P7 primers, the solid support may be contacted with the template to be amplified under conditions which permit hybridization (or annealing—such terms may be used interchangeably) between the template and the immobilized primers. The template is usually added in free solution under suitable hybridization conditions, which will be apparent to the skilled reader. Typically, hybridization conditions are, for example, 5xSSC at 40° C. Solid-phase amplification can then proceed. The first step of the amplification is a primer extension step in which nucleotides are added to the 3′ end of the immobilized primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilized on the solid support and binding. Further rounds of amplification (analogous to a standard PCR reaction) lead to the formation of clusters or colonies of template molecules bound to the solid support.

### Substrates

Additional aspect of the present disclosure relates to a substrate for sequencing, comprising:
template polynucleotides attached to a surface of the substrate through a first plurality of bonding sites via covalent or noncovalent bonding; and
a second plurality of bonding sites for capturing primer oligonucleotides;
wherein the surface of the substrate comprises a plurality of patterned nanowells, and wherein at least 50%, 55%, 60%, 65%, 70%, 75%, 80%, 85%, 90%, 95%, 98%, or 99% of the nanowells is each occupied with each occupied with one single template polynucleotide. In further embodiments, the substrate is prepared according to the low salt seeding method described herein.

In any embodiments of the substrate, the first plurality of bonding sites and/or the second plurality of bonding sites may be the same as those described in the low salt seeding method.

In some embodiments, the substate comprises patterned surfaces. For example, the substrate may make use of solid supports comprised of a substrate or matrix (e.g. glass slides, polymer beads etc.) which has been “functionalized”, for example by application of a layer or coating of an intermediate material comprising reactive groups which permit covalent attachment to biomolecules, such as surface primer oligonucleotides. Examples of such supports include, but are not limited to, a substrate such as glass. In such embodiments, the biomolecules (e.g. surface primers) may be directly covalently attached to the intermediate material but the intermediate material may itself be non-covalently attached to the substrate or matrix (e.g. the glass substrate). Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide. In one embodiment, the solid support may be functionalized with azido groups. In further embodiments, the azido groups may be introduced by an intermediate material such as a PAZAM coating.

In other embodiments, the solid support may be “functionalized” by application of a layer or coating of an intermediate material comprising groups that permit non-covalent attachment to biomolecules. In such embodiments, the groups on the solid support may form one or more of ionic bonds, hydrogen bonds, hydrophobic interactions, π-π interactions, van der Waals interactions and host-guest interactions, to a corresponding group on the biomolecules (e.g. polynucleotides). The interactions formed between the group on the solid support and the corresponding group on the biomolecules may be configured to cause immobilization or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing. In one embodiment, the solid support may be functionalized to introduce avidin bonding sites (e.g. streptavidin).

In other embodiments, the solid support may be “functionalized” by application of an intermediate material comprising groups that permit attachment via metal-coordination bonds to biomolecules. In such embodiments, the groups on the solid support may include ligands (e.g. metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilization or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

When referring to immobilization or attachment of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In certain embodiments of the invention, covalent attachment may be preferred; in other embodiments, attachment using non-covalent interactions may be preferred; in yet other embodiments, attachment using metal-coordination bonds may be preferred. However, in general the molecules (e.g. nucleic acids) remain immobilized or attached to the support under the conditions in which it is intended to use the support, for example in applications requiring nucleic acid amplification and/or sequencing. When referring to attachment of nucleic acids to other nucleic acids, then the terms “immobilized” and “hybridized” are used herein, and generally refer to hydrogen bonding between complementary nucleic acids.

If the amplification is performed on beads, either with a single or multiple extendable primers, the beads may be analyzed in solution, in individual wells of a microtiter or picotiter plate, immobilized in individual wells, for example in a fiber optic type device, or immobilized as an array on a solid support. The solid support may be a planar surface, for example a microscope slide, wherein the beads are deposited randomly and held in place with a film of polymer, for example agarose or acrylamide.

### Sequencing Applications

Some embodiments are directed to methods of detecting an analyte using a substrate with a patterned surface prepared by the methods described herein. In some embodiments, the analyte is selected from nucleic acids, polynucleotides, proteins, antibodies, epitopes to antibodies, enzymes, cells, nuclei, cellular organelles, or small molecule drugs. In one embodiment, the analyte is a polynucleotide. In one embodiment, the detecting includes determining a nucleotide sequence of the polynucleotide.

Some embodiments that use nucleic acids can include a step of amplifying the nucleic acids on the substrate. Many different DNA amplification techniques can be used in conjunction with the substrates described herein. Exemplary techniques that can be used include, but are not limited to, polymerase chain reaction (PCR), rolling circle amplification (RCA), multiple displacement amplification (MDA), or random prime amplification (RPA). In particular embodiments, one or more oligonucleotide primers used for amplification can be attached to a substrate (e.g. via the azido silane layer). In PCR embodiments, one or both of the primers used for amplification can be attached to the substrate. Formats that utilize two species of attached primer are often referred to as bridge amplification because double stranded amplicons form a bridge-like structure between the two attached primers that flank the template sequence that has been copied. Exemplary reagents and conditions that can be used for bridge amplification are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference.

PCR amplification can also be carried out with one amplification primer attached to a substrate and a second primer in solution. An exemplary format that uses a combination of one attached primer and soluble primer is emulsion PCR as described, for example, in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference. Emulsion PCR is illustrative of the format and it will be understood that for purposes of the methods set forth herein the use of an emulsion is optional and indeed for several embodiments an emulsion is not used. Furthermore, primers need not be attached directly to substrate or solid supports as set forth in the ePCR references and can instead be attached to a gel or polymer coating as set forth herein.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and US 2007/0099208 A1, each of which is incorporated herein by reference. Primers used for RCA can be in solution or attached to a gel or polymer coating.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a gel or polymer coating.

In particular embodiments a combination of the above-exemplified amplification techniques can be used. For example, RCA and MDA can be used in a combination wherein RCA is used to generate a concatemeric amplicon in solution (e.g. using solution-phase primers). The amplicon can then be used as a template for MDA using primers that are attached to a substrate (e.g. via a gel or polymer coating). In this example, amplicons produced after the combined RCA and MDA steps will be attached to the substrate.

Substrates of the present disclosure that contain nucleic acid arrays can be used for any of a variety of purposes. A particularly desirable use for the nucleic acids is to serve as capture probes that hybridize to target nucleic acids having complementary sequences. The target nucleic acids once hybridized to the capture probes can be detected, for example, via a label recruited to the capture probe. Methods for detection of target nucleic acids via hybridization to capture probes are known in the art and include, for example, those described in U.S. Pat. Nos. 7,582,420; 6,890,741; 6,913,884 or 6,355,431 or U.S. Pat. Pub. Nos. 2005/0053980 A1; 2009/0186349 A1 or 2005/0181440 A1, each of which is incorporated herein by reference. For example, a label can be recruited to a capture probe by virtue of hybridization of the capture probe to a target probe that bears the label. In another example, a label can be recruited to a capture probe by hybridizing a target probe to the capture probe such that the capture probe can be extended by ligation to a labeled oligonucleotide (e.g., via ligase activity) or by addition of a labeled nucleotide (e.g. via polymerase activity).

In some embodiments, a substrate described herein can be used for determining a nucleotide sequence of a polynucleotide. In such embodiments, the method can comprise the steps of (a) contacting a substrate-attached polynucleotide/copy polynucleotide complex with one or more different type of nucleotides in the presence of a polymerase (e.g., DNA polymerase); (b) incorporating one type of nucleotide to the copy polynucleotide strand to form an extended copy polynucleotide; (c) perform one or more fluorescent measurements of one or more the extended copy polynucleotides; wherein steps (a) to (c) are repeated, thereby determining the sequence of the substrate-attached polynucleotide.

Nucleic acid sequencing can be used to determine a nucleotide sequence of a polynucleotide by various processes known in the art. In a preferred method, sequencing-by-synthesis (SBS) is utilized to determine a nucleotide sequence of a polynucleotide attached to a surface of a substrate (e.g. via any one of the polymer coatings described herein). In such a process, one or more nucleotides are provided to a template polynucleotide that is associated with a polynucleotide polymerase. The polynucleotide polymerase incorporates the one or more nucleotides into a newly synthesized nucleic acid strand that is complementary to the polynucleotide template. The synthesis is initiated from an oligonucleotide primer that is complementary to a portion of the template polynucleotide or to a portion of a universal or non-variable nucleic acid that is covalently bound at one end of the template polynucleotide. As nucleotides are incorporated against the template polynucleotide, a detectable signal is generated that allows for the determination of which nucleotide has been incorporated during each step of the sequencing process. In this way, the sequence of a nucleic acid complementary to at least a portion of the template polynucleotide can be generated, thereby permitting determination of the nucleotide sequence of at least a portion of the template polynucleotide.

Flow cells provide a convenient format for housing an array that is produced by the methods of the present disclosure and that is subjected to a sequencing-by-synthesis (SBS) or other detection technique that involves repeated delivery of reagents in cycles. For example, to initiate a first SBS cycle, one or more labeled nucleotides, DNA polymerase, etc., can be flowed into/through a flow cell that houses a nucleic acid array made by methods set forth herein. Those sites of an array where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference in its entirety.

In some embodiments of the above-described method, which employ a flow cell, only a single type of nucleotide is present in the flow cell during a single flow step. In such embodiments, the nucleotide can be selected from the group consisting of dATP, dCTP, dGTP, dTTP, and analogs thereof. In other embodiments of the above-described method which employ a flow cell, a plurality different types of nucleotides are present in the flow cell during a single flow step. In such methods, the nucleotides can be selected from dATP, dCTP, dGTP, dTTP, and analogs thereof.

Determination of the nucleotide or nucleotides incorporated during each flow step for one or more of the polynucleotides attached to the polymer coating on the surface of the substrate present in the flow cell is achieved by detecting a signal produced at or near the polynucleotide template. In some embodiments of the above-described methods, the detectable signal comprises an optical signal. In other embodiments, the detectable signal comprises a non-optical signal. In such embodiments, the non-optical signal comprises a change in pH at or near one or more of the polynucleotide templates.

Applications and uses of substrates of the present disclosure have been exemplified herein with regard to nucleic acids. However, it will be understood that other analytes can be attached to a substrate set forth herein and analyzed. One or more analytes can be present in or on a substrate of the present disclosure. The substrates of the present disclosure are particularly useful for detection of analytes, or for carrying out synthetic reactions with analytes. Thus, any of a variety of analytes that are to be detected, characterized, modified, synthesized, or the like can be present in or on a substrate set forth herein. Exemplary analytes include, but are not limited to, nucleic acids (e.g., DNA, RNA or analogs thereof), proteins, polysaccharides, cells, antibodies, epitopes, receptors, ligands, enzymes (e.g., kinases, phosphatases or polymerases), small molecule drug candidates, or the like. A substrate can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

In some embodiments, analytes can be distributed to features on a substrate such that they are individually resolvable. For example, a single molecule of each analyte can be present at each feature. Alternatively, analytes can be present as colonies or populations such that individual molecules are not necessarily resolved. The colonies or populations can be homogenous with respect to containing only a single species of analyte (albeit in multiple copies). Taking nucleic acids as an example, each feature on a substrate can include a colony or population of nucleic acids and every nucleic acid in the colony or population can have the same nucleotide sequence (either single stranded or double stranded). Such colonies can be created by cluster amplification or bridge amplification as set forth previously herein. Multiple repeats of a target sequence can be present in a single nucleic acid molecule, such as a concatemer created using a rolling circle amplification procedure. Thus, a feature on a substrate can contain multiple copies of a single species of an analyte. Alternatively, a colony or population of analytes that are at a feature can include two or more different species. For example, one or more wells on a substrate can each contain a mixed colony having two or more different nucleic acid species (i.e. nucleic acid molecules with different sequences). The two or more nucleic acid species in a mixed colony can be present in non-negligible amounts, for example, allowing more than one nucleic acid to be detected in the mixed colony.

What is claimed is:

1. A method of preparing a substrate for sequencing, comprising:
contacting a first buffer solution comprising template polynucleotides with a surface of the substrate, wherein the surface of the substrate comprises a first plurality of bonding sites for capturing template polynucleotides and a second plurality of bonding sites for capturing primer oligonucleotides; and
attaching the template polynucleotides to the surface of the substrate by forming covalent bonding or non-covalent bonding between the template polynucleotides and the first plurality of the bonding sites of the surface;
wherein the first buffer solution comprises a total concentration of salt or salts of about 100 mM or less.

2. The method of claim 1, wherein the template polynucleotides are single-stranded polynucleotides.

3. The method of claim 1 or 2, wherein the first plurality bonding sites of the surface comprises non-covalent bonding sites.

4. The method of claim 3, wherein the non-covalent bonding sites comprise streptavidin.

5. The method of claim 4, wherein each of the template polynucleotides comprises a biotin moiety.

6. The method of claim 1 or 2, wherein the first plurality bonding sites of the surface comprise covalent bonding sites.

7. The method of claim 6, wherein the covalent bonding sites comprise amino bonding sites, carboxy bonding sites, thiol bonding sites, aldehyde bonding sites, azido bonding sites, hydroxy bonding sites, transcyclooctene bonding sites, norbornene bonding sites, cyclooctyne bonding sites, oxoamine bonding sites, SpyTag bonding sites, Snap-tag bonding sites, CLIP-tag bonding sites, or proteins with N-terminus recognized by sortase, or combinations thereof.

8. The method of claim 7, wherein each of the template polynucleotides comprises a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling.

9. The method of any one of claims 1 to 8, wherein the concentration of the template polynucleotides in the first buffer solution is about 10 pM to about 2000 pM, about 100 pM to about 1000 pM, about 200 pM to about 500 pM, or about 250 pM to about 350 pM.

10. The method of any one of claims 1 to 9, wherein the first buffer solution has a pH of about 3.5 or less.

11. The method of any one of claims 1 to 10, wherein the first buffer solution further comprises one or more crowding agents.

12. The method of any one of claims 1 to 11, further comprising:
contacting a second buffer solution comprising the primer oligonucleotides with the surface of the substrate; and
attaching the primer oligonucleotides to the surface of the substrate by forming covalent bonding or non-covalent bonding between the primer oligonucleotides and the second plurality of the bonding sites of the surface;
wherein the second buffer solution comprises a total concentration of salt or salts of about 250 mM or greater.

13. The method of claim 12, wherein the primer oligonucleotides comprise a first type of primer oligonucleotide and a second type of primer oligonucleotides.

14. The method of claim 13, wherein the primer oligonucleotides comprise P5 primer sequence and P7 primer sequence.

15. The method of claim 12 or 13, wherein the second plurality bonding sites of the surface comprises comprise covalent bonding sites.

16. The method of claim 15, wherein the second plurality bonding sites of the surface comprise amino bonding sites, carboxy bonding sites, thiol bonding sites, aldehyde bonding sites, azido bonding sites, hydroxy bonding sites, transcyclooctene bonding sites, norbornene bonding sites, cyclooctyne bonding sites, oxoamine bonding sites, SpyTag bonding sites, Snap-tag bonding sites, CLIP-tag bonding sites, or proteins with N-terminus recognized by sortase, or combinations thereof.

17. The method of claim 16, wherein each of the plurality of primer oligonucleotides comprises a NHS ester moiety, an aldehyde moiety, an imidoester moiety, a pentofluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a dibenzocyclooctyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling.

18. The method of any one of claims 12 to 17, further comprising amplifying the template polynucleotides.

19. The method of any one of claims 1 to 18, wherein the surface of the substrate comprises a plurality of patterned nanowells.

20. The method of claim 19, wherein at least 50% of the nanowells is each occupied with only one cluster of template polynucleotide or only one dominant cluster of template polynucleotide.

21. A substrate for sequencing, comprising:
template polynucleotides attached to a surface of the substrate through a first plurality of bonding sites via covalent or noncovalent bonding; and
a second plurality of bonding sites for capturing primer oligonucleotides;
wherein the surface of the substrate comprises a plurality of patterned nanowells, and wherein at least 50% of the nanowells is each occupied with a single template polynucleotide.
